(19) United States
(12) Patent Application Publication
(10) Pub. No.: US 2009/0088615 A1
(43) Pub. Date: Apr. 2, 2009

(54) Indwelling Fiber Optic Probe for Blood Glucose Measurements

(76) Inventors: Mark Ries Robinson, Albuquerque, NM (US); Russell Abbink, Sandia Park, NM (US); Alan Ross, Albuquerque, NM (US)

Correspondence Address: V. Gerald Grafe, esq., P.O. Box 2689, Corrales, NM, 87048, US

(21) Appl. No.: 12/241,221

(22) Filed: Sep. 30, 2008

Related U.S. Application Data
(60) Provisional application No. 60/976,775, filed on Oct. 1, 2007.

(51) Int. Cl. A61B 5/1459; A61M 5/142

(52) U.S. Cl. 600/316; 604/66

(57) ABSTRACT

An indwelling fiber optic probe can be used to make in vivo blood glucose measurements through a central venous catheter. The fiber optic probe can operate in the near-infrared spectral region. The optical measurement can be backscattering, transmission, or a combination of both, depending on the optical configuration.

### CROSS-REFERENCE TO RELATED APPLICATION

This application claims the benefit of U.S. Provisional Application No. 60/976,775, “Indwelling Fiber Optic Probe for Blood Glucose Measurements,” filed Oct. 1, 2007, which is incorporated herein by reference.

### FIELD OF THE INVENTION

The present invention relates to glucose monitoring and, in particular, to an indwelling fiber optic probe that can be used for blood glucose measurements.

### BACKGROUND OF THE INVENTION

Diabetes mellitus is an endocrine metabolic disorder resulting from a lack of insulin that affects over 170 million people worldwide. Improved glucose sensing would enable improved glycemic control, thereby delaying the onset of serious medical complications associated with diabetes. An indispensable tool for both diabetic and critically ill patients is a reliable blood glucose measurement method. Most diabetic patients currently use self-monitoring via finger pricking and test strips to check their blood glucose level and adjust their insulin dosage to maintain normal blood glucose concentrations. Although such self-monitoring of blood glucose has been an indispensable tool for diabetes therapy, it is fraught with difficulties. Frequent finger pricking is painful, costly, and inconvenient for the patient. As a result of this invasiveness, many diabetics frequently skip self-monitoring tests. Further, tight control of blood glucose is difficult to achieve without frequent glucose measurements. Intermittent measurements can be influenced by other changes in the patient's physical state and testing conditions. Glucose fluctuations during the day, and particularly during the night, are often missed using self-monitoring techniques.

One desirable system for the management of glycemia is a continuous in-vivo glucose monitoring method that could be coupled with an automated insulin pump for active closed-loop control of glucose level. In-vivo glucose sensing devices being developed comprise both implanted and noninvasive sensors. Invasive devices can be implanted intravascularly in the blood stream or interstitially under the skin, since the concentration of glucose within the interstitial fluid correlates with the glucose concentration in the blood. Alternate invasive technologies to measure blood glucose remove blood from the body for interrogation and analysis. This blood might be discarded or infused back into the body. Typically, if blood is infused, saline is also used which adds more fluid to the body. Noninvasive glucose sensors measure glucose concentrations in vivo without direct physical contact between the sensor and the biological fluid. Such noninvasive sensors are patient friendly and can eliminate biocompatibility problems. Most in-vivo glucose sensors are based on electrochemical or colorimetric/photometric detection techniques.

Colorimetric and photometric approaches can be used to monitor glucose levels directly. For example, vibrational spectroscopic approaches can use the unique vibration transitions within the glucose molecule. Vibrational spectroscopies include Raman spectroscopy and absorption spectroscopy in the mid- and near-infrared spectral regions. Raman spectroscopy can measure fundamental vibrational bands, but sensing applications have been hampered by the presence of a strong background fluorescence signal and low signal-to-noise ratio due to an inherently weak Raman signal. Glucose is a relatively simple monosaccharide molecule with strong and distinctive absorption features in the mid-infrared (MIR) region. Unfortunately, water and other non-glucose metabolites, such as proteins, amino acids, urea, fatty acids, and triglycerides also strongly absorb in the MIR.

Therefore, emphasis has shifted to the detection of molecular absorptions in the near-infrared (NIR) spectral region corresponding to combinations and overtones of fundamental glucose molecular vibrations. The strong OH and CH stretch bands in the 2900 to 3600 cm⁻¹ MIR region can generate overtone and combination bands in the 700 to 1700 nm NIR region. Additional glucose-specific combinations of CH stretch and ring deformation bands occur at wavelengths greater than 2000 nm. Although the glucose absorptions in the NIR are unique, they are weaker and broader than the fundamental bands and also overlap with bands from other tissue components, such as water, fat, and hemoglobin. Therefore, multivariate chemical analysis methods can be used to extract glucose-specific spectral information.

Noninvasive optical sensors can use optical radiation to probe regions of tissue, such as the finger, tongue, or ear, and extract glucose concentration from a measured spectrum. Noninvasive NIR sensors use the “optical window” in the near infrared in which the absorbance by human biological tissue is lower compared to the visible or ultraviolet regions. However, these noninvasive NIR sensors can have measurement difficulties due to the weak glucose absorption peaks, relatively low glucose concentrations in human tissue, multiple interferences with non-glucose metabolites, variations in tissue hydration, blood flow, environmental temperature, and light scattering.

Fiber optic probes can be used for minimally invasive optical sensors. See Utzinger and Richards-Kortum, J. Biomedical Optics 8(1), 121 (2003), which is incorporated herein by reference. Fiber optic probes provide a flexible optical interface between a light source, spectrometric detector, and the tissue being interrogated so that the light source and detector can be located remote from the patient. A dual-fiber arrangement can be used for separate illumination and collection. The collection fiber optic can transport the remitted light from the interrogated tissue to the spectrometer.

An individual optical fiber typically comprises a core, a cladding, and a protective jacket. Fibers can be packed into bundles to provide a larger optical active area. Coupling optics can adapt the f-number of the light source to the numerical aperture of the fiber to optimize irradiance into the fiber. The ends of a fiber can be cleaved or polished for optimal coupling. Further, the exit surface can be beveled to deflect the light output or input. Probe geometries can comprise side-looking probes that use obliquely polished ends to deflect the output of the fiber in respect to the fiber axis, probes with diffuser tips to provide homogeneous illumination of large areas in canals and on surfaces, and refocusing probes that refocus the illumination or collection beam path to decrease or increase the sample volume illuminated.

Probe assemblies have also been used for indwelling light scattering spectroscopy for biomedical applications. See U.S. Pat. No. 6,366,726 to Wach et al., which is incorporated herein by reference. In particular, Raman spectroscopy can provide a means for chemical identification. With Raman spectroscopy, incident laser light is transmitted over an optical fiber to the sample medium and the Raman-scattered is returned via the same or another fiber to a spectrometer for analysis. The Raman-scattered light is color shifted from the incident illumination beam by a specific amount related to molecular band vibrations. Further, the intensity of the shifted return light correlates with the chemical concentration. However, in-vivo Raman spectroscopy using flat face, parallel illumination and collection fiber probes has been hampered by the inefficiency of scattered light collection. Wach describes several approaches to direct and manipulate illumination and receptivity zones to improve Raman-scattered light collection efficiency. These approaches include varying the numerical apertures of the illumination and collection fibers, use of confocal optics, bending the tips of the fibers to increase the overlapping region, shaping the fibers' end faces to create a refractive surface to manipulate the illumination and collection zones, and manipulating the light with light-shaping structures within the confines of the fiber assembly's internal structure. Therefore, the probe can be designed to have selective sensitivity to the Raman scattering signal by delivering light at one angle and collecting light at the appropriate angle to maximize the response. However, sensing applications based on Raman spectroscopy have been hampered by the silica-Raman effect and fiber fluorescence and the inherently low weak Raman signal.

Therefore, a need remains for an in-vivo continuous glucose monitoring method that uses an indwelling fiber optic probe to measure glucose concentration or presence in the near-infrared spectral region.

### SUMMARY OF THE INVENTION

The present invention provides an indwelling fiber optic probe that can be used to make blood glucose measurements through a central venous catheter. The probe can also be used to measure other metabolites, such as blood gases, lactate, hemoglobin and urea.

The indwelling probe does not require blood to be removed from the body, thus simplifying the instrumentation, reducing blood loss and possible saline infusion. The overhead time of removing the blood from the body is also eliminated, allowing for a longer measurement time, which can enable improvements in signal-to-noise ratio through signal averaging.

The fiber optic probe can operate in the NIR spectral region, for example the region from 3000 cm⁻¹ to 9000 cm⁻¹. The optical measurement can be backscattering, transmission, or a combination of both, depending on the optical configuration.

The probe can have at least one illumination and at least one collection fiber, although the same fiber or fibers can be used for both illumination and collection. The illumination input to the probe can be from a light source that is suitable for use in NIR. The output to the detector can interface to an FTIR spectrometer. The end of the probe that interfaces with the blood can be optimized by changing several parameters which will affect the signal detected.

The fiber optic probe can be a disposable and inserted into a catheter or it can be integral to a catheter. A non-disposable probe can also be inserted into a disposable catheter.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 shows a schematic illustration of a glucose monitoring device comprising an indwelling fiber optic probe.

FIGS. 2A-2F show schematic illustrations of example optical configurations for the indwelling fiber optic probe.

FIGS. 3A and 3B show fiber optic probes comprising a catheter containing a plurality of illumination and collection fibers.

FIGS. 4A-4C show three types of fiber optic probe constructions.

FIGS. 5A and 5B show a fiber optic probe for collecting a reference saline background measurement.

FIGS. 6A and 6B show fiber optic probe configurations for an auxiliary fiber optic measurement.

### DETAILED DESCRIPTION OF THE INVENTION

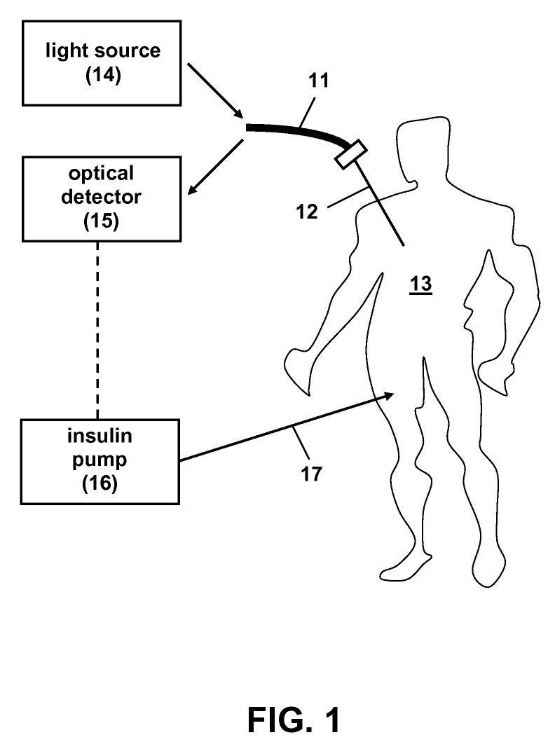

FIG. 1 shows a schematic illustration of a glucose monitoring device comprising an indwelling fiber optic probe according to the present invention. A non-disposable illumination and collection fiber optic 11 can be coupled to a short disposable indwelling fiber optic probe 12 that can be integrated into a catheter that is inserted into a patient 13. The illumination portion of the fiber optic 11 can be connected to an ex vivo light source 14 for delivery of the illumination light to the patient tissue to be analyzed. The light source can be a near-infrared (NIR) light source, such as a thermal source, a tunable laser, or multiple lasers at selected wavelengths. The collection portion of the fiber optic 11 can be connected to an ex-vivo optical detector 15 for the detection of the tissue spectrum in the NIR spectral region. For example, the fiber optic probe 12 can be inserted intravascularly into blood tissue. Glucose in the blood can affect the detected transmitted or reflected tissue spectrum by absorption of light at the overtone and combination band wavelengths. For example, the detector 15 can comprise a Fourier transform infrared (FTIR) spectrometer. The detector 15 can further use signal processing methods, such as multivariate spectral analysis algorithms, to analyze the glucose-specific spectral features of the detected tissue spectrum. The device can further comprise an insulin pump 16 for infusing insulin 17 into the patient 13 in closed-loop response to the blood glucose measurement.

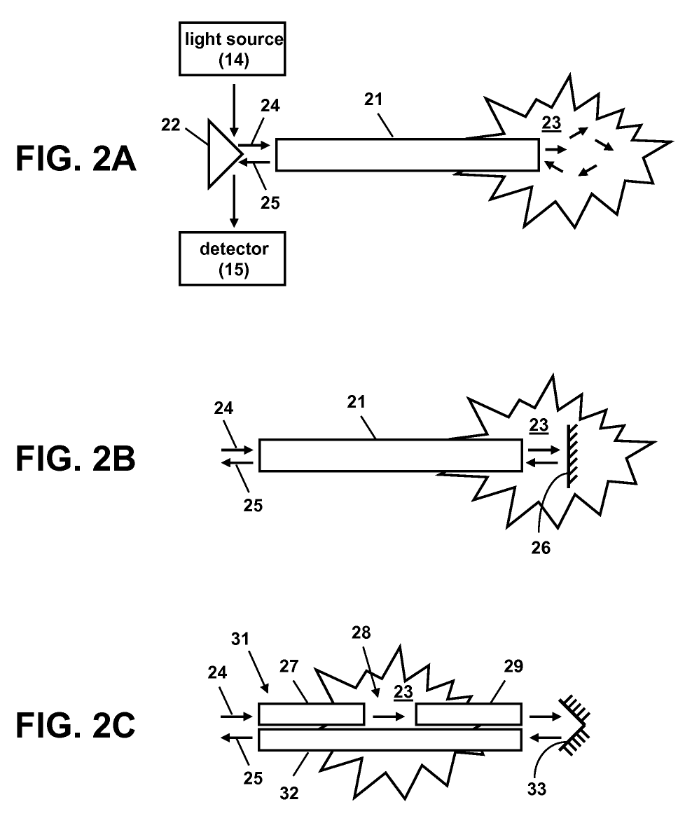

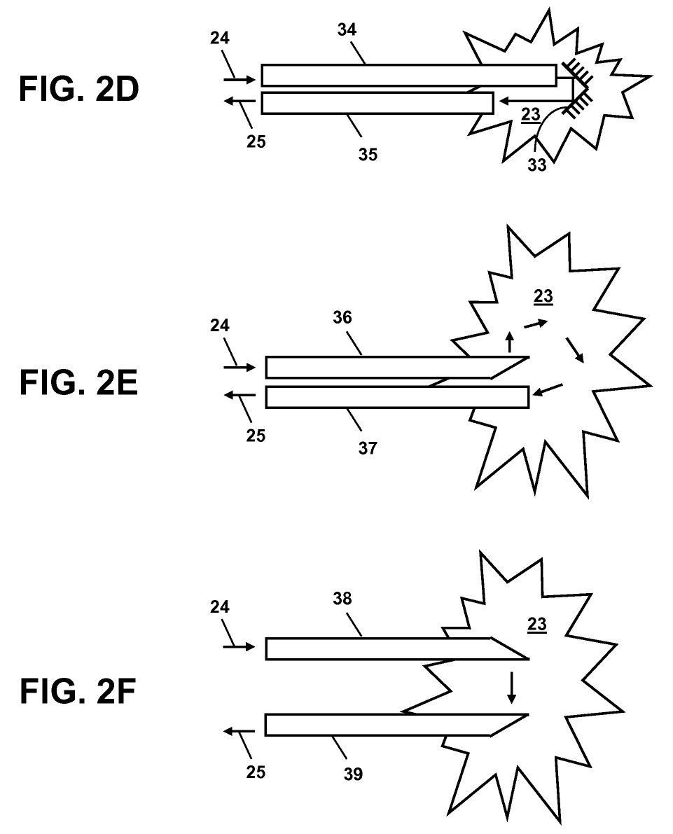

The fiber optic probe can comprise various illumination and collection optical configurations comprising one or more optical fibers having flat faced or shaped ends, and external optical elements, such as micromirrors and microlenses, to optimize the illumination and collection characteristics of the sample volume. Further, the numerical aperture, core and cladding materials, geometry, size, and arrangement and number of optical fibers can be chosen to optimize the delivery of light to and from the blood sample and to enable biocompatibility of the indwelling probe. The optical fibers can be contained in a catheter that can be inserted into a patient's tissue. FIGS. 2A-2F are schematic illustrations of some example optical configurations.

FIG. 2A shows an example configuration comprising a single optical fiber 21 that can be used for both the illumination and the collection of light that is diffusely reflected or scattered by the patient's blood. Near-infrared light 24 is provided by the light source 14 and is coupled into the proximal end of the optical fiber 21 by a reflecting wedge 22. The distal end of the optical fiber 21 can have a flat face for illuminating a blood sample 23 with the light 24 from the light source 14. For example, the fiber 21 can be integrated into a catheter that is inserted into the patient's blood stream and the blood can be sampled through a hole in the catheter. The light 24 can be scattered by the blood sample 23 and the scattered light 25 can be collected through the flat face of the distal end of the fiber 21. The collected light 25 is returned by the optical fiber 21 to the wedge 22 which reflects the collected light to the optical detector 15. Alternatively, lenses or similar optical elements can be used to couple the illumination light and collected light 25 into and out of the fiber. Alternatively, one or more separate illumination fibers can be used to illuminate the blood sample and one or more collection fibers can be used to collect the scattered light and return the collected light to the detector.

FIG. 2B shows an example optical configuration comprising a single optical fiber 21 for both the illumination and the collection of light that is both transmitted through and scattered by the patient's blood. NIR light 24 from a light source enters the proximal end and exits the flat face of the distal end of optical fiber 21 to illuminate the blood sample 23. Both transmitted and scattered light is collected by the fiber 21. Light that is transmitted through the sample is reflected by a flat mirror 26 at the distal end of the probe and is coupled, along with the scattered light, into the distal end of the fiber 21 as collected light 25. The optical path length to and from the end of the fiber to the mirror can be chosen to maximize the glucose signal. The collected light 25 is returned to an optical detector by the optical fiber 21. Alternatively, one or more separate illumination fibers can be used to illuminate the blood sample and one or more collection fibers can be used to collect the transmitted and scattered light and return the collected light to the detector.

FIG. 2C shows an example optical configuration comprising an illumination fiber 31 and a parallel collection fiber 32 that collects the illumination light that is transmitted by the patient's blood. The illumination fiber 31 can have a gap 28 separating a proximal portion 27 and the distal portion 29 of the fiber. NIR light 24 from a light source enters the proximal end of the proximal portion 27 of the fiber. A hole in the side wall of a catheter that contains the fibers can allow blood to flow across the gap 28 in the fiber. The illumination light 24 exits the flat face end of proximal portion 27 of the fiber and is transmitted through the blood sample 23 in the gap 28. The length of the gap 28 can be chosen to provide a suitable glucose signal based upon the penetration depth of the light 24 in the sample 23. The transmitted light enters the flat face entrance of the distal portion 29 of the fiber, exits the flat face end of the distal portion 29, and is reflected by a turning mirror 33 into a collection fiber 32. The collected light 25 is returned to an optical detector by the collection fiber 32. Additionally or alternatively, a gap can be provided in the collection fiber for transmission of the return light through the blood sample.

FIG. 2D shows an example optical configuration comprising an illumination fiber 34 and a parallel collection fiber 35 that collects the illumination light that is transmitted by the patient's blood. The distal end of the illumination fiber 34 is butted up to or in close proximity to the turning mirror 33. The distal end of the collection fiber 35 is retracted from the mirror 33 such that most of the optical pathlength is between the mirror 33 and the distal end of the collection fiber 35. This pathlength can be chosen to provide a suitable glucose signal based upon the penetration depth of the light 24 in the sample 23. The transmitted light enters the flat face distal end of the collection fiber 35 and the collected light 25 is returned to an optical detector by the collection fiber 35. Alternatively, the distal end of the collection fiber can be butted up to the turning mirror and the distal end of the illumination fiber can be retracted from the mirror to provide the desired optical pathlength.

FIGS. 2E and 2F show example optical configurations that use side-looking optical fibers having beveled ends for the collection of both scattered and transmitted light. In FIG. 2E, illumination light 24 from an NIR light source exits the beveled face of the side-looking distal end of an illumination fiber 36 and is scattered by the blood sample 23. Some of the scattered light is collected by the flat-face distal end of a collection fiber 37 and the collected light 25 is returned to an optical detector. In FIG. 2F, illumination light 24 from a side-looking illumination fiber 38 is collected by a side-looking collection fiber 39 and the collected light 25 is returned to an optical detector. This optical configuration preferentially collects light that is transmitted through the blood sample 23.

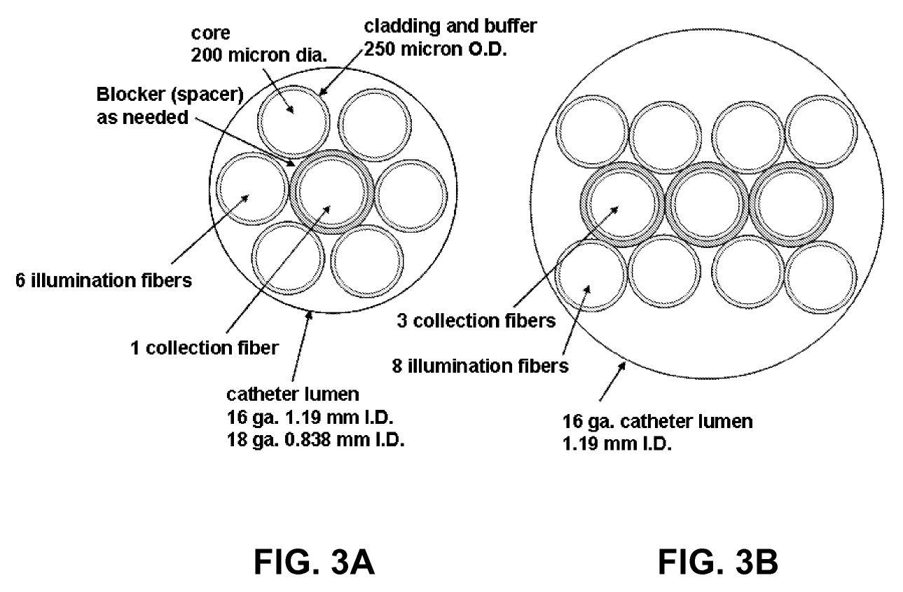

The small dimensions of optical fibers allow multiple illumination and collection fibers to be bundled into a single catheter. FIGS. 3A and 3B show examples of illumination and collection fiber geometries that are compatible with 16 and 18 ga. catheters. The catheter lumen can comprise at least one illumination fiber and at least one collection fiber. The spacing between the illumination and collection fibers, the number of fibers, and the size of the fibers can be optimized to improve the detected signal. The fibers can be step- or gradient-index fibers comprising a high refractive index core and a lower refractive index cladding for efficient guiding of near-infrared light. The core of the fibers can comprise an optical material, such as glass or silica, that is transparent in the near-infrared. The examples shown are for optical fibers with a 200 micron core with a cladding to provide a 250 micron outside diameter fiber.

FIG. 3A shows an example fiber optic probe comprising a catheter containing six parallel illumination fibers surrounding a central collection fiber. The collection fiber can have an opaque blocker or spacer on the outside of the cladding layer to inhibit cross-talk with the illumination fibers. As examples, the catheter lumen can be 16 ga. (1.19 mm inside diameter) or 18 ga. (0.838 mm inside diameter).

FIG. 3B shows an example fiber optic probe comprising a catheter having two planes of four illumination fibers each surrounding a central plane of three collection fibers. As an example, the fibers can be contained in a 16 ga. catheter lumen having a 1.19 mm inside diameter.

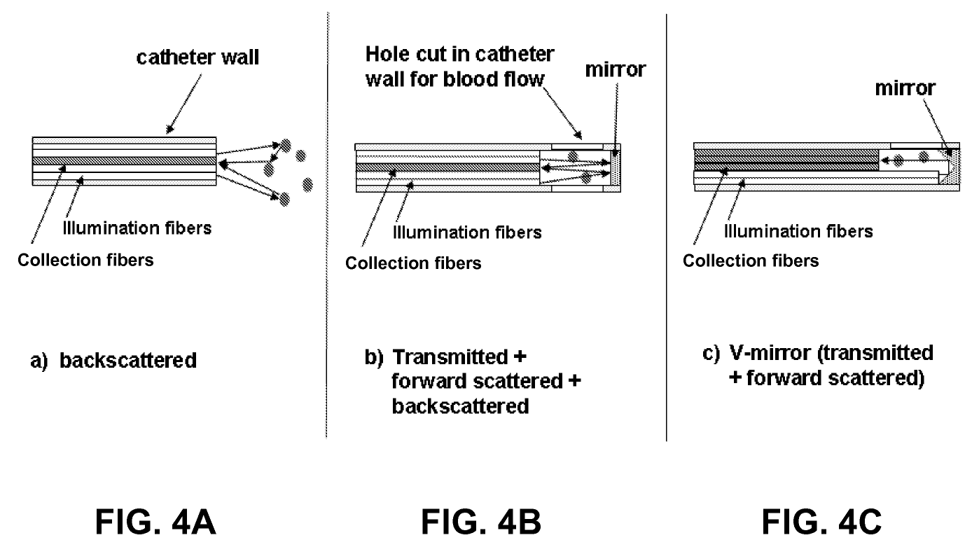

FIGS. 4A-4C show example probe constructions that comprise the optical configurations shown in FIGS. 2A, 2B, and 2D, respectively.

FIG. 4A shows an example probe wherein light from peripheral illumination fibers is backscattered by the blood and the backscattered light is collected at a central collection, or detector, fiber. The fibers can be contained in a catheter having an open distal end exposed to the blood sample. The number of illumination and collection fibers, and their arrangement, controls the pathlength and magnitude of signal detected. The shape of the probe tip and individual fibers can be designed to provide a suitable signal for detection.

FIG. 4B shows an example probe wherein transmitted, forward scattered, and backscattered light is collected by a central collection fiber. Blood flows across the probe through a hole cut in the catheter wall. The distance from the fibers to the mirror allows control of the optical pathlength. Backscattered light (not reflected by the mirror) can also be collected.

FIG. 4C shows an example probe optimized for a transmission measurement which collects transmitted light only. The illumination fibers are butted up to the turning mirror and the collection fibers are retracted from the mirror. In this configuration, the optical pathlength is controlled by the spacing of the collection fibers to the turning mirror. Blood flows across the probe through a hole in the catheter wall.

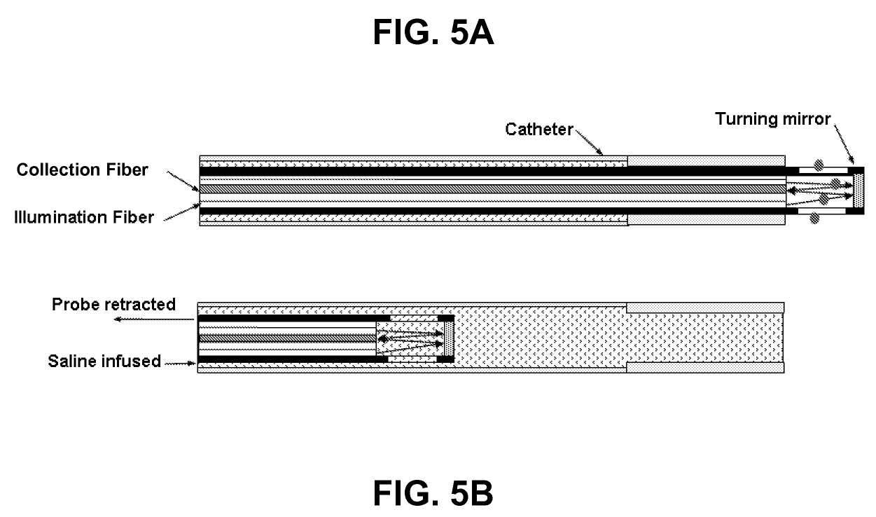

The optical probe can be configured to enable a background reference measurement. FIGS. 5A and 5B show example probes for collecting a reference saline background measurement. FIG. 5A shows the probe in a sample-measuring configuration, similar to the optical configurations shown in FIGS. 2B and 4B. In FIG. 5B, the fiber probe is shown retracted within the catheter lumen. The catheter is sealed and saline is infused into the catheter housing. The infused saline can flow around the probe, enabling a reference saline background measurement.

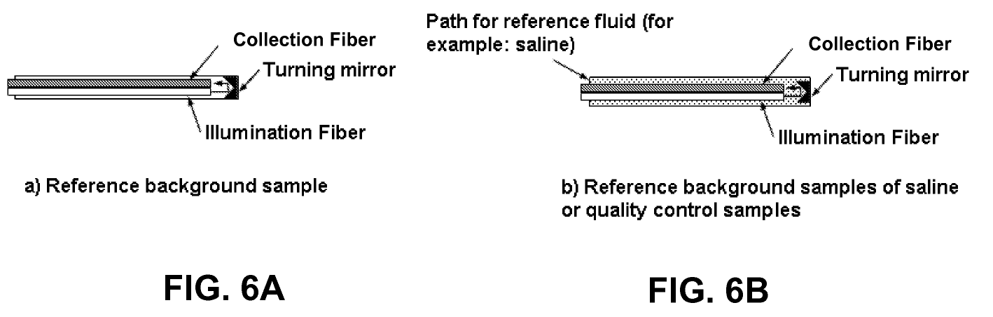

FIGS. 6A and 6B show example probe configurations for an auxiliary fiber optic measurement. FIG. 6A shows an example reference background probe that uses illumination and detection fibers and a turning mirror, but without the sample. The extra channel of information from the reference background probe can be used to compensate for spectra effects resulting from bending of multimode fiber optics. The background probe can run along side the sample probe fibers that are used for the blood measurement, but would not be indwelling. The probe can also provide a reference background measurement to compensate for the stability of the sample probe or to simply monitor the health of the sample probe. FIG. 6B shows an example auxiliary fiber probe that incorporates a fluid measurement, such as saline, within the housing of the probe. This probe can be used as another method of background correction for the sample measurement probe.

The present invention has been described as an indwelling fiber optic probe. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1. An indwelling fiber optic probe, comprising at least one optical fiber having a proximal end and a distal end, wherein illumination light from a near-infrared light source is coupled into the proximal end and directed to the distal end of the fiber and wherein the distal end is inserted into a patient tissue and wherein light from the tissue is collected by the distal end of the at least one optical fiber and returned to the proximal end of the fiber as collected light.

2. The probe of claim 1, further comprising a catheter containing the at least one optical fiber.

3. The probe of claim 2, wherein the catheter comprises a hole that allows the tissue to access the distal end of the at least one optical fiber.

4. The probe of claim 1, further comprising an optical detector for detecting a tissue spectrum of the collected light.

5. The probe of claim 4, wherein the optical detector comprises a Fourier transform infrared spectrometer.

6. The probe of claim 1, wherein the at least one optical fiber comprises glass or silica.

7. The probe of claim 1, wherein the collected light comprises backscattered or transmitted light from the tissue.

8. The probe of claim 1, wherein the tissue comprises blood.

9. The probe of claim 1, wherein the tissue comprises a metabolite.

10. The probe of claim 9, wherein the metabolite comprises glucose.

11. The probe of claim 10, wherein the collected light includes light at wavelengths correlated with glucose concentration or presence.

12. The probe of claim 11, further comprising an insulin pump for infusing insulin into the patient in response to the glucose concentration or presence.

13. The probe of claim 9, wherein the distal end of the at least one fiber is shaped to optimize a near-infrared metabolite signal of the collected light.

14. The probe of claim 1, further comprising an optical element proximate the distal end of the at least one optical fiber for coupling the collected light back into the at least one fiber.

15. The probe of claim 14, where the optical element comprises a mirror or a lens.

16. The probe of claim 1, wherein the at least one optical fiber comprises an illumination fiber and a collection fiber.

17. The probe of claim 16, wherein at least one of the illumination or collection fibers comprises a gap between a proximal portion and a distal portion of the fiber and wherein the gap is exposed to the patient tissue so that the scattered or transmitted light from the tissue is collected by the distal portion of the fiber.

18. The probe of claim 16, further comprising a turning mirror proximal the distal end of the illumination fiber for reflecting the collected light back into the collection fiber, wherein the spacing between the distal ends of the illumination and collection fibers and the mirror is chosen to provide a suitable tissue signal of the collected light.

19. The probe of claim 16, wherein at least one of the illumination or collection fibers comprises a side-looking fiber.

20. The probe of claim 19, wherein both the illumination fiber and the collection fiber comprise side-looking fibers.
